(19) United States
(12) Patent Application Publication
(10) Pub. No.: US 2007/0082404 A1
(43) Pub. Date: Apr. 12, 2007

(54) Method of Measuring Fluoride in Fluxes Using the Fluoride Ion-Selective Electrode

(76) Inventors: Jerry L. Yeager, Monroeville, NJ (US); Kandalam V. Ramanujachary, Deptford, NJ (US)

Correspondence Address: rudoler & derosa llc;ATTN: DOCKET CLERK, 2 BALA PLAZA,, SUITE 300, BALA CYNWYD, PA, 19004, US

(21) Appl. No.: 11/456,815

(22) Filed: Jul. 11, 2006

Related U.S. Application Data
(60) Provisional application No. 60/698,085, filed on Jul. 11, 2005.
(60) Provisional application No. 60/765,469, filed on Feb. 3, 2006.

(51) Int. Cl. G01N 33/00

(52) U.S. Cl. 436/124

(57) ABSTRACT

A method for analyzing fluoride in fluxes comprises the steps of placing the flux into an aqueous solution. Chelating agents are added to the aqueous flux solution to form a chelated aqueous flux solution. The chelating agents are selected from the group consisting of ammonium citrate dibasic, ammonium tartrate dibasic, citric acid, ethylene diamine, disodium-EDTA solution, and sodium chloride. And, a potentiometric analysis is performed on the chelated aqueous flux solution using the plurality of control standard solutions for determining the fluoride content of the flux.

### CROSS-REFERENCE TO RELATED APPLICATIONS

The present application claims the benefit of U.S. Provisional Application No. 06/698,085 filed on Jul. 11, 2005 and U.S. Provisional Application No. 60/765,469 filed on Feb. 2, 2006, which are incorporated herein by reference.

### STATEMENT REGARDING FEDERALLY SPONSORED RESEARCH OR DEVELOPMENT

The present invention was not developed with the use of any Federal Funds, but was developed independently by the inventor.

### FIELD OF THE INVENTION

The invention relates to measuring fluorides in fluxes, and particularly to measuring fluorides in fluxes using the Fluoride Ion-Selective Electrode (FISE).

### BACKGROUND OF THE INVENTION

Fluxes are a substance that is routinely used in metal refining that facilitates fusion of minerals or metals, removal of impurities in the melt, and prevention of the formation of oxides. Electroslag Refining Flux (ESR flux), in particular, is a fused solid mixture of calcium fluoride, lime, magnesia, alumina, silica and other metallic oxides used in the production of high quality alloys and superalloys. ESR flux is important to the metals industry where it is used in all applications where it is used in applications requiring metal strength and thermal resistance. Many types of ESR flux have been developed over the years in the industry, with specific mixtures of the constituent compounds being tailored to each application. For these fluxes to be effective in their processes, the constituent mixture must adhere to specific compositions determined by the specific application(s) of the product(s) being produced. Thus, the need for analysis of these fluxes for their composition is of obvious importance.

Prior Art Methods of Fluoride Detection

Verifying the chemical composition of fluxes is often a difficult and time consuming process. The mixture of oxides, fluorides, silicates, and the like presents analytical challenges when quantitative analyses for major constituents and trace impurities are sought. Calcium fluoride (fluorspar) is one of the most important constituents of ESR flux; it is typically present in concentrations ranging from 30 to 70% (by mass), CaF₂ fluxes being the most common. However, in the presence of other calcium species and other metal oxides, such as Al₂O₃, MgO, and TiO₂, the CaF₂ is difficult to quantify. A common and practical approach is to measure the concentration of fluoride in the flux and then calculate the stoichiometrically equivalent amount of calcium fluoride. The difficulty in this approach lies in the complex and variable composition of flux, that prevents the use of some traditional analytical methods to determine its fluoride content.

Several wet methods for fluoride analysis exist, but these are generally time consuming, expensive, operator dependent, and require modifications to be useful for analysis of EST flux. One of the oldest of these methods is the Berzelius Method, which involves fusion of the flux sample, precipitation of constituent metals, and, finally, precipitation of fluoride as calcium fluoride, which must be washed, dried and weighted. This method is reliable and accurate, but involves toxic reagents, is time-consuming, and operator dependent. Another method involves the distillation from sulfuric acid or perchloric acid solution. This method is also time-consuming and is subject to interferences from aluminum and silica, both of which are present in ESR flux in high quantities. One method used by American Flux & Metal, called the Foote Method (a variant pyrohydrolytic method developed by the, now defunct, Foote Mineral Company) is an example of a method that has been used for analysis of fluoride content shown in the flowchart in FIG. 1. This procedure is time consuming, expensive, high-maintenance, and subject to numerous operator errors. In addition, much of the apparatus for this method must be custom-made.

In spite of their widespread use in metals analyses, more conventional laboratory instrumental techniques, such as Inductively Coupled Plasma-Optical Emission Spectrometry (ICP-OES) and Atomic Absorption-Graphite Furnace (AA-GF), have inherent limitations that prevent their application to detecting fluoride ions. Analysis of the solid flux for fluoride content has been conducted in the past with expensive instrumentation such as X-ray diffraction, X-ray Fluorescence (XRF), Electron Micro-Probe Analysis (EMPA), and/or Glow Discharge Mass Spectrometry (GD-MS). X-ray techniques are considered more suitable for this type of analysis, but numerous difficulties remain a concern when quantitative determinations are needed.

The method of the present invention is a rapid, low-cost, accurate, and reproducible method to analyze for fluoride in fluxes using equipment that is readily available to analytical laboratories. Potentiometric techniques offer utility for a cost-effective, reliable and timely fluoride analysis and most types of samples do not require extensive preparation. In addition, sample solutions with high turbidity are rarely problematic with potentiometric methods. The potentiometric method of the present invention is validated with ESR flux samples and samples simulating the composition of ESR fluxes (i.e., control samples). The examples of the present invention described herein are directed to an analytical approach for determining total fluoride, reported as % CaF₂ (by mass) (fluorspar), in ESR fluxes; however, it should be understood that the invention has wider applicability to the detection of fluoride in other fluxes and other materials.

### BRIEF SUMMARY OF THE INVENTION

Various other features and attendant advantages of the present invention will become more fully appreciated and better understood when considered in conjunction with the accompanying figures.

A method for analyzing fluoride in fluxes, such as electroslag refining flux, comprises the steps of placing the flux into an aqueous solution. Chelating agents are added to the aqueous flux solution to form a chelated aqueous flux solution. The chelating agents are selected from the group consisting of ammonium citrate dibasic, ammonium tartrate dibasic, citric acid, ethylene diamine, disodium-EDTA solution, and sodium chloride. And, a potentiometric analysis is performed on the chelated aqueous flux solution using the plurality of control standard solutions for determining the fluoride content of the flux. Optionally, the pH of the chelated aqueous flux solution is held between 8 and 9. A plurality of control standards in a chelated aqueous solution may also be prepared, each comprising a varying amount of fluoride for checking the calibration of test instruments used in the analysis step.

In one preferred embodiment, the chelating agents are added in the following order: ammonium citrate dibasic, then ammonium tartrate dibasic, then citric acid, then ethylene diamine, then disodium-EDTA solution, and then sodium chloride.

In another preferred embodiment, the step for placing the flux into an aqueous solution comprises using crystalline sodium tetraborate decahydrate and anhydrous sodium carbonate in the ratio of 2:5:7 mass ratios.

In another preferred embodiment the flux solution is diluted with DI water and the dilution factor may be 1 g of flux to 200 ml of solvent.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

In the drawings:

FIG. 1 depicts a prior art flowchart for pyrohydrolytic fluorine analysis (Foote Method);

FIG. 2 depicts a flowchart for direct potentiometric fluoride analysis method (FISE Method) of the present invention;

FIG. 3 depicts a typical calibration plot of the potentiometer using the five listed calibration standards of the present invention; and

FIG. 4 depicts a plot of the deviation from linearity between theoretical fluoride content, Foote Method, and the direct potentiometry method of the present invention.

### DETAILED DESCRIPTION OF THE INVENTION

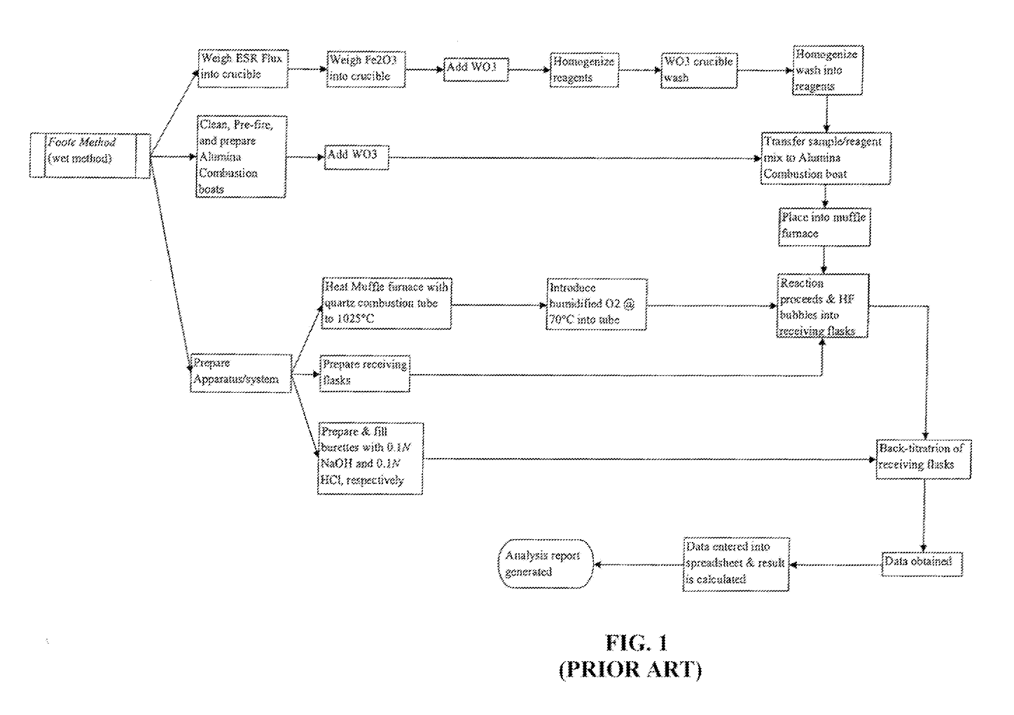

The invention relates to a preferred method of measuring fluoride in fluxes using the fluoride ion-selective electrode. FIG. 1 depicts a flow chart depicting the steps for carrying out the potentiometric fluoride determination method 10 of the present invention.

The present invention relates to the use of potentiometric methods in an effort to develop a low-cost, rapid and reproducible method for F⁻ion estimation. Potentiometric techniques offer utility for a cost-effective, reliable and timely fluoride analysis because measurements are quick, inexpensive, and most types of samples do not require extensive preparation. In addition, samples containing suspended particulates in solution are not problematic with potentiometric methods. Methods for potentiometric determination of fluoride in a variety of matrices, such as drinking water and blood samples, have been extensively investigated utilizing a FISE.

Because an FISE responds to F⁻ion activity in aqueous solution, cations in the solution matrices that form metal complexes with fluoride ions (reducing the availability of free fluoride ions) are potential negative interferences to the FISE technique. In many common applications, such as drinking water analyses, these interferences are minimal, due to the low levels of cations present. However, in the case of ESR flux, interferences are cause for concern due to the abundance of interfering polyvalent cations such as Fe⁺³, Al⁺³, Mn⁺⁴, Ti⁺⁴, Mg⁺², and Ca⁺² in the flux matrix. Varying concentrations of these cations make it impossible to predict and mathematically correct for these interferences. The key to allowing the persistence of free F⁻in solution is to “tie up” interfering cations by adding ligands to the solution that have a greater affinity for the metal cations than does fluoride, releasing fluoride anions to be detected by the FISE.

Amino-polycarboxylic acids such as EDTA, DPTA, CDTA, and NTA are well known chelating agents that form stable coordination spheres with metal cations. In addition, polycarboxylic acids such as citric acid, tartaric acid, and salicylic acid, have been used as chelating agents. Based on availability, cost, solubility, and chelating effectiveness, a combination of chelating agents (EDTA, ammonium citrate, and ammonium tartrate) are used in the present method to complex metal cations. An excess of these complexing agents masks the effect of cations on fluoride activity and drives complex formation equilibria toward coordination molecule formation. In Table 1, the overall formation constants (log K_{f}) for many of the complexes formed by these chelants with metals commonly found in ESR flux are shown.

TABLE 1
Overall Formation Constants (log Kᵣ) for
Metal Complexes When All Ligand Sites Are Deprotonated^{a}

| metal ion | EDTA | ethylenediamine | citrate | tartrate |
|---|---|---|---|---|
| aluminum | 16.11 | | 20.0 | |
| calcium | 11.0 | | 4.68 | 9.01 |
| chromium(II) | | 13.94 | | |
| chromium(III) | 23 | | | |
| copper(II) | 18.7 | 20.00 | 14.2 | 4.78 |
| iron(II) | 14.33 | 9.70 | 15.5 | |
| iron(III) | 24.23 | | 25.0 | 7.49 |
| lanthanum | 16.34 | | 9.45 | 3.06 |
| lead(II) | 18.3 | | 6.50 | 3.78 |
| magnesium | 8.64 | | 3.29 | 1.38 |
| manganese(II) | 13.8 | 5.67 | 3.67 | |
| nickel(II) | 18.56 | 18.33 | 14.3 | |
| titanium(IV) | 17.3 | | | |
| zirconium | 19.40 | | | |

^{a}The elements listed are metals, expected to be present in ESR flux formulations. Source: Adapted with Permission from ref 4. Copyright 1995 McGraw-Hill.

The reaction was carried out under conditions of relatively high pH (8-9) to maintain a high conditional formation constant of EDTA and drive complex-formation reactions forward. To analyze ESR flux via potentiometric techniques, the flux 12 must first be placed into aqueous solution 14. Under normal conditions, ESR flux is almost completely insoluble with acid. A flux fusion using crystalline sodium tetraborate decahydrate and anhydrous sodium carbonate, in 2:5:7 mass ratios (1 g of sample:2.5 g of Na₂B₄O₇•10H₂O:3.5 g of Na₂CO₃), is used to create solid matrixes that were digestible by dilute nitric acid. The resulting solution is diluted with deionized (DI) water to make a solution containing 1 g of ESR flux IN 200 mL of solution. All water used for dilutions was 18 MΩ DI H₂O. This procedure is repeated for the control standard and all other flux samples analyzed. The control standard that is typically used for the analyses is a mixture of ACS grade reagents from Alfa Aesar certified at >99.99% purity (metals basis) and was prepared by weighing 0.5982 g of 99.99% purity (by mass) CaF₂, 0.1003 g of 99.76% (after subtracting water content) purity (by mass) CaO, 0.0999 g of 99.998% purity (by mass) MgO, 0.1002 g of 99.999% purity (by mass) Al₂O₃, 0.0497 g of 99.995% purity (by mass) SiO₂, and 0.0498 g of 99.995% purity (by mass) TiO₂ into a platinum 30 mL crucible and fusing the mixture as previously mentioned. The calculated mass of fluoride in the control standard is 0.2911 g in the sample, yielding a concentration of 0.0766 M F⁻in 200 mL of prepared solution.

Chelating agents 15 are added to the ESR flux solution 14. To prepare the chelated solution, 4 mL of ESR flux solution, 20 mL of 2 M ammonium citrate dibasic, 10 mL of 2 M ammonium tartrate dibasic, 10 mL of 2 M citric acid, 4 mL of ethylene diamine, 25 mL of 50%_{(m/v)} disodium-EDTA solution, and 5 mL of 2 M sodium chloride were added to a 100 mL volumetric flask, in this specific order, with DI water making up the balance. This order of the additions appears to be important because it appears that precipitate may form when the order is altered. The final theoretical fluoride concentration of the control standard is 3.064×10⁻³ M when prepared in this manner. All reagents used were ACS reagent grade. The pH of these chelated solutions was held between pH 8 and 9, because chelation of metal cations by EDTA is dependent on the pH of the solution.

For fluxes that contained relatively high aluminum content (>30% Al₂O₃ by mass) such as F30 and F40, the pH of flux solutions is adjusted closer to pH 8.0 by lowering the amount of ethylene diamine added. This is done because the high stability of the aluminum-citrate & aluminum-EDTA complexes, as shown in Table 1, does not require as high of a pH under the solution conditions to drive aluminum-complex formation. In general, this is the case for the majority of metals having high stability constants with both EDTA and citrate (the primary chelants), however Cu²⁺, Fe^{2+, 3+}, and Ni³⁺ are not present in appreciable quantities in the tested fluxes. Only Al³⁺ followed by this exception in the samples tested. A pH closer to 9.0 in these sample solutions is potentially unfavorable and skewed results high by several percent.

In contrast, for samples having high calcium fluoride content (≧60% (by mass) CaF₂), the pH is adjusted as close to pH 9.0 as possible by adding additional ethylene diamine (due to the lower stability of calcium-chelant complexes) to increase the masking effects of the chelants on metal cation interferences. In this case, higher OH⁻ content helped to achieve better masking of calcium-fluoride interactions, yielding good results.

These adjustments are made to minimize the possible interference of excess OH⁻ ions on electrode performance. In addition, the disodium-EDTA solution (obtained from VWR International) contains additional sodium hydroxide that helped buffer pH. The ammonium salts also serve to buffer the solution, minimizing the effect that variations in flux matrixes have on the pH of the chelated solutions and the sodium chloride adjusts the solutions' ionic strength.

ACS reagent grade 0.1000M NaF is used as the source of fluoride for the calibration standards 30 since Na+ cations do not form stable complexes with F⁻ anions in aqueous solution. Five calibration standards are prepared with concentrations ranging from 5.00*10⁻⁴M F⁻ to 4.00*10⁻³M F⁻ to encompass a broad range of expected fluoride content. The chelating agents used for the ESR solutions are also used for these standards, so as to match the matrices between samples and standards. The amount of ethylene diamine used in 3 mL for each standard; slightly less than that used for ESR flux samples because CaF₂ is not present in these calibration standards. In addition, 4 mL of fused flux blank solution containing 3.5 g (±0.01 g) anhydrous sodium carbonate, 2.5 g (±0.01 g) crystalline sodium tetraborate decahydrate, 0.5001 g of CaO, 0.0505 g MgO, 0.4002 g Al₂O₃, 0.0199 g SiO₂, and 0.0307 g TiO₂ (reagents previously described), fused and brought to 200 mL volume with deionized H₂O, is added to each standard to better match the matrix to that of the chelated ESR flux solutions. Finally, detection of fluoride activity in the standards is not as dependent on pH as in ESR flux solutions, since the concentrations of metal cations in ACS grade NaF is negligible.

With the chelated solution 29 obtained and the potentiometer calibrated 30, analyses on the chelated solution are performed 40. From the analyzes, data is obtained 42 and which may be entered into a spreadsheet or otherwise calculated 44. Finally, an analysis report is generated 46.

The method used for analysis is a direct-read method for potentiometric analysis utilizing the Accumet Excel25 potentiometer/pH bench-top meter (Dual Channel) and a BNC LaF³ glass-body Fluoride Ion-Selective Combination Electrode. All samples and standards stayed at room temperature (22° C. during analyses, which was monitored via ATC probe. The pH meter is calibrated via a three-point calibration using pH 4, pH 7, and pH 10 certified pH buffers. Samples and standards are each allowed to equilibrate for 3 to 5 minutes according to standard potentiometric procedure. Assuming the raw materials other than CaF₂ used in the manufacture of the flux contain only trace levels of fluoride, observed fluoride ion concentration can be directly correlated to total CaF₂ content.

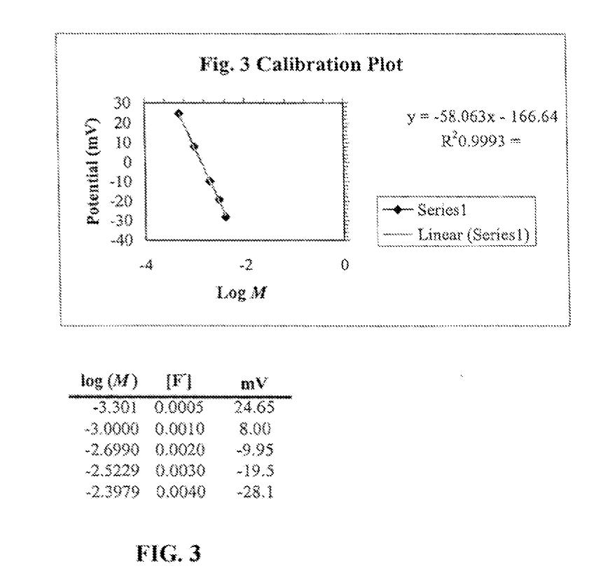

A typical observed calibration curve is depicted in FIG. 3. The slope of −57.6 mV(decade)⁻¹ for this curve indicates excellent Nernstian response for the electrode at 22° C.

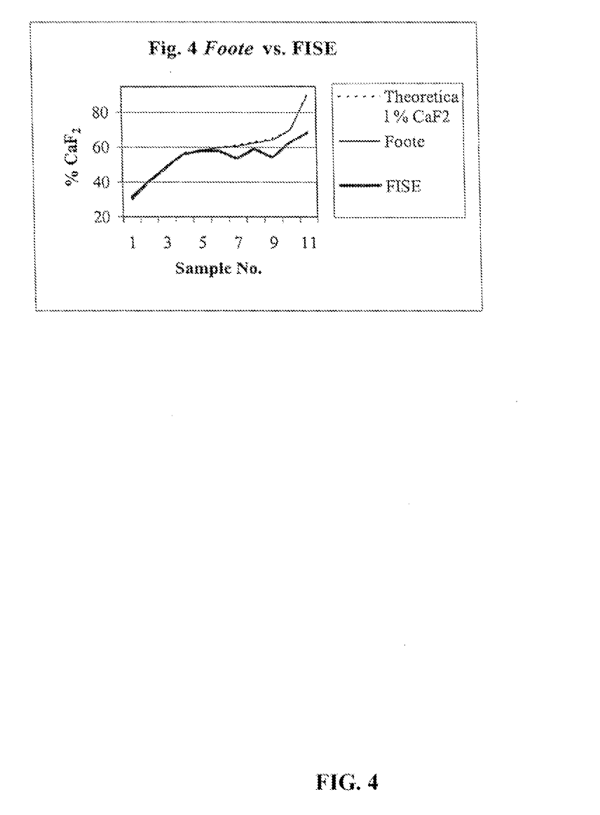

Table 2, below, compares the results of analyses on 12 flux samples by the FISE method of the present invention with analyses by the Foote method. Between one and eight replicate analyses (the same for each method) are typically performed on each sample. The relative standard deviation is also shown for the FISE method results. For each sample, FIG. 4 graphs the average result obtained by each method, showing error bars of ±one standard deviation where multiple runs were made. FISE results are read directly from the potentiometer in units of molarity and converted into % CaF₂₍ₜₒₜₐₗ₎ (by mass).

TABLE 2
Comparison of Analytical Results for
ESR Flux by FISE and Foote Methods

| flux^{a} | N^{b} | Footeₘₑₐₙ^{c} | FISEₘₑₐₙ^{d} | RSD_{FISE}^{e} (%) |
|---|---|---|---|---|
| F30 | 5 | 30.7 | 31.1 | 2.8 |
| F40 | 8 | 40.0 | 40.6 | 2.4 |
| F48 | 4 | 47.9 | 48.9 | 1.9 |
| F56 | 6 | 56.4 | 56.1 | 2.0 |
| F58 | 4 | 58.6 | 58.8 | 3.9 |
| F60 | 4 | 59.2 | 60.7 | 5.2 |
| Control | 6 | 59.3 | 58.9 | 1.8 |
| F61 | 4 | 60.4 | 60.3 | 0.6 |
| F63 | 4 | 62.5 | 61.7 | 2.8 |
| F65 | 4 | 64.3 | 54.4 | 1.9 |
| F70^{f} | 1 | 69.8 | 62.8 | |
| F90^{f} | 1 | 89.5 | 68.5 | |

^{a}Flux fomulations with the designation “F” followed by the expected mean % CaF₂ content.
^{b}Number of measurements performed for each flux designation.
^{c}The mean % CaF₂ of the results obtained for each flux designation by the Foote method.
^{d}The mean % CaF₂ of the results obtained for each flux designation by the FISE method.
^{e}Relative standard deviation (% RSD) for FISE results.
^{f}Only one replicate was run for F70 and F90.

As shown in Table 2 and FIG. 4, excellent agreement was obtained between the two methods for samples containing <63% (by mass) CaF₂ as analyzed by the Foote Method. Above this limit, FISE results showed little further increase in measured fluoride content, and are lower than results by the Foote Method.

Because calcium has a relatively low formation constant with the chelants used relative to the other cations present in ESR flux solution (Table 1), it is more difficult to mask its detrimental effects on fluoride activity. Therefore, an excess of chelants is used in this work to reduce these calcium-fluoride interactions allowing the FISE to better detect fluoride ions. Adjustments to pH failed to improve detection of fluoride when CaF₂ content was >63% (by mass), indicating a point of diminishing returns when compensating for higher calcium content in fluxes using pH adjustment.

Standard deviations of the analytical results by the FISE and Foote methods were not significantly different for any of the ten flux samples measured in replicate, when statistically tested using an F-test. Relative standard deviations of individual measurements were roughly 2% to 3% for both methods, indicating good precision across all concentrations for both methods (Table 2). When CaF₂ content was ≦63% (by mass), the difference between the mean CaF₂ values obtained from the Foote and FISE methods (Table 2) were acceptable and different by a maximum of 1.5%.

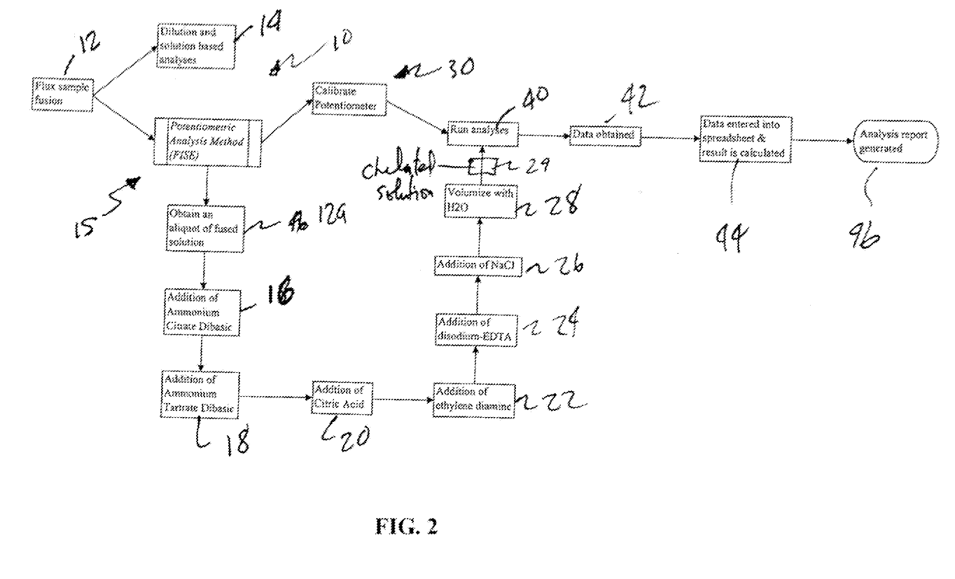

As can be seen by the results, reliable analysis of fluoride content in ESR flux is quite possible using the analytical method of the present invention. Comparing the flowcharts in FIGS. 1 and 2 for the two methods, we can see that the proposed method is more streamlined and straightforward than the Foote Method. The average analysis time for this procedure is short compared to other techniques, as well. Many samples can be run daily; calibration of the meter takes roughly 20 minutes, and each sample, thereafter, takes only 3 to 5 minutes to analyze. Comparatively speaking, the Foote Method takes 1 to 4 hours in preparation time, and 25 minutes for each sample analyzed. With the proposed FISE method, preparation of the samples is somewhat involved, but the fused solution can be used for analysis of other flux constituents. In addition, the initial setup cost and annual reagents costs for this method are far less than all other techniques available, costing about 25% less than the Foote Method, and >90% less than X-ray methods. The reagents are easily and inexpensively obtained, have low toxicity, and the consumable parts for the instrumentation are rugged and inexpensive. Even a laboratory on a strict budget with minimal staff can find utility in this method.

It is evident from the results of this work that the rapid fusion with subsequent chelation and fluoride ion determination described in this procedure is suitable for analysis for total fluoride content in many fluxes.

The use of EDTA, ethylene diamine, tartrates, and citrates for chelation allows for reliable analysis of most types of fluxes used in the industry. It is feasible that this method could be applied to other fluoride containing minerals as well, such as cryolite. In its current form, the method of the present invention remains a quick, effective and reliable technique for analysis of fluoride content in fluxes.

The benefits described above are not necessary to the invention, are provided by way of demonstration and are not intended to in any way limit the invention. For example, alternative combinations of chelating agents may improve the method's utility with fluxes containing higher concentrations of calcium and magnesium. In addition, speciating the coordinating compounds that are formed in the chelated ESR solution may yield further beneficial results.

I/we claim:

1. A method for analyzing fluoride in fluxes comprising the steps of:
placing the flux into an aqueous solution;
adding a plurality of chelating agents to the aqueous flux solution to form a chelated aqueous flux solution; and
performing a potentiometric analysis on the chelated aqueous flux solution for determining the fluoride content of the flux.

2. The method for analyzing fluoride in fluxes of claim 1 wherein the chelating agents are selected from the group consisting of ammonium citrate dibasic, ammonium tartrate dibasic, citric acid, ethylene diamine, disodium-EDTA solution, and sodium chloride.

3. The method for analyzing fluoride in fluxes of claim 1 wherein the chelating agents are ammonium citrate dibasic, ammonium tartrate dibasic, citric acid, ethylene diamine, disodium-EDTA solution, and sodium chloride.

4. The method for analyzing fluoride in fluxes of claim 3 wherein the chelating agents are added in the following order: ammonium citrate dibasic, then ammonium tartrate dibasic, then citric acid, then ethylene diamine, then disodium-EDTA solution, and then sodium chloride.

5. The method for analyzing fluoride in fluxes of claim 1 wherein the step for placing the flux into an aqueous solution comprises using crystalline sodium tetraborate decahydrate and anhydrous sodium carbonate.

6. The method for analyzing fluoride in fluxes of claim 5 wherein the ratio of flux, crystalline sodium tetraborate decahydrate, and anhydrous sodium carbonate is 2:5:7 mass ratios.

7. The method for analyzing fluoride in fluxes of claim 1 wherein the step for placing the flux into an aqueous solution further comprises the step of diluting the flux solution with DI water wherein the dilution factor is about 1 g of flux to 200 ml of solvent.

8. The method for analyzing fluoride in fluxes of claim 1 wherein the pH of the chelated aqueous flux solution is between about 8 and 9.

9. The method for analyzing fluoride in fluxes of claim 1, further comprising the step of preparing a plurality of control standards in an chelated aqueous solution each comprising a varying amount of fluoride for checking the calibration of test instruments used in the analysis step.

10. The method for analyzing fluoride in fluxes of claim 1 wherein the flux is an electroslag refining flux.

11. The method for analyzing fluoride in fluxes, comprising the steps of:
placing the flux into an aqueous solution using crystalline sodium tetraborate decahydrate and anhydrous sodium carbonate;
adding a plurality of chelating agents to the aqueous flux solution to form a chelated aqueous flux solution, the chelating agents are selected from the group consisting of ammonium citrate dibasic, ammonium tartrate dibasic, citric acid, ethylene diamine, disodium-EDTA solution, and sodium chloride;
performing a potentiometric analysis on the chelated aqueous flux solution for determining the fluoride content of the flux.

12. The method for analyzing fluoride in fluxes of claim 11 wherein the chelating agents are added in the following order: ammonium citrate dibasic, then ammonium tartrate dibasic, then citric acid, then ethylene diamine, then disodium-EDTA solution, and then sodium chloride.

13. The method for analyzing fluoride in fluxes of claim 11 wherein the ration of flux, crystalline sodium tetraborate decahydrate, and anhydrous sodium carbonate is 2:5:7 mass ratios.

14. The method for analyzing fluoride in fluxes of claim 13 wherein the ratio of flux, crystalline sodium tetraborate decahydrate, and anhydrous sodium carbonate is 2:5:7 mass ratios.

15. The method for analyzing fluoride in fluxes of claim 11 wherein the step for placing the flux into an aqueous solution further comprises the step of diluting the flux solution with DI water wherein the dilution factor is 1 g of flux to 200 ml of solvent.

16. The method for analyzing fluoride in fluxes of claim 14 wherein the step for placing the flux into an aqueous solution further comprises the step of diluting the flux solution with DI water wherein the dilution factor is 1 g of flux to 200 ml of solvent.

17. The method for analyzing fluoride in fluxes of claim 11 wherein the pH of the chelated aqueous flux solution is between about 8 and 9.

18. The method for analyzing fluoride in fluxes of claim 16 wherein the pH of the chelated aqueous flux solution is between about 8 and 9.

19. The method for analyzing fluoride in fluxes of claim 11, further comprising the steps of preparing a plurality of control standards in a chelated aqueous solution, each comprising a varying amount of fluoride for checking the calibration of test instruments used in the analysis step.

20. The method for analyzing fluoride in fluxes of claim 11 wherein the flux is an electroslag refining flux.
